(19) United States
(12) Patent Application Publication
(10) Pub. No.: US 2025/0186796 A1
(43) Pub. Date: Jun. 12, 2025

(54) ELECTROMAGNETIC DEVICE AND METHOD FOR TREATING CANCERS AND TUMORS

(71) Applicant: LEV P. DVORKIN, K. BIALEK (IL)

(72) Inventor: LEV P. DVORKIN, K. BIALEK (IL)

(21) Appl. No.: 19/059,730

(22) Filed: Feb. 21, 2025

Related U.S. Application Data
(63) Continuation-in-part of application No. 17/521,337, filed on Nov. 8, 2021.
(60) Provisional application No. 63/140,114, filed on Jan. 21, 2021.

(51) Int. Cl. A61N 1/40

(52) U.S. Cl. CPC A61N 1/403

(57) ABSTRACT

The electromagnetic device and method for treating cancers or tumors changes the metabolism of cells, helping to stop proliferation of cancers or tumor cells inside or on the body. The device includes an RF frequency generator for generating an RF carrier signal, a low frequency generator for generating a modulating signal, a modulator for frequency modulating the RF carrier signal with the modulating signal to produce a frequency-modulated output signal, and an antenna with a parabolic trough reflector for directing the modulated signal to a patient's body.

### CROSS-REFERENCE TO RELATED APPLICATIONS

This application is a continuation-in-part (CIP) of U.S. patent application Ser. No. 17/521,337, filed on Nov. 8, 2021, which claims the benefit of provisional application 63/140,114, filed on Jan. 21, 2021, the entire contents of each of which are incorporated herein by reference.

### BACKGROUND

### 1. Field

The disclosure of the present patent application relates to RF therapy devices, and particularly to an electromagnetic device and method for treating cancers and tumors.

### 2. Description of the Related Art

According to the World Health Organization, a study in 2018 indicated that every fifth man and every sixth woman will get cancer at some stage of their life. The annual cost of treating skin cancers in the U.S. is estimated at $8.1 billion, including about $4.8 billion for non-melanoma skin cancers, and $3.3 billion for melanoma. In the U.S., more than 9,500 people are diagnosed with skin cancer every day. More than two people die of the disease every hour. Basal cell carcinoma (BCC) is the most common form of skin cancer. An estimated 4.3 million cases of BCC are diagnosed in the U.S. each year. Squamous cell carcinoma (SCC) is the second most common form of skin cancer. More than 1 million cases of SCC are diagnosed in the U.S. each year. Organ transplant patients are approximately 100 times more likely than the general public to develop squamous cell carcinoma. Current figures suggest that more than 15,000 people die of SCC in the U.S. each year, more than twice as many as from melanoma. Neuroblastoma is by far the most common cancer in infants (younger than 1 year old). There are about 700 to 800 new cases of neuroblastoma each year in the United States. This number has remained about the same for many years. The National Institutes of Health (NIH) estimates that 80% of all women will develop uterine fibroids (myomas) at some point during their lives. Because many women don't experience any symptoms, it's possible that the incidence of uterine fibroids is even higher. Fibroids are considered benign or noncancerous, but can be painful.

Some current RF therapy devices for treating cancer use amplitude-modulated RF signals. While these devices are somewhat effective, they are limited in their performance, and their ability to treat neuroblastoma, squamous cell carcinoma, and benign (noncancerous) tumors, such as myoma, has not been thoroughly evaluated.

Thus, an electromagnetic device and method for treating cancers and tumors solving the aforementioned problems is desired.

### SUMMARY

An electromagnetic device and method for treating cancers and tumors changes the metabolism of cells, helping to stop proliferation of tumors and cancer cells inside or on the body and providing epigenetic reprogramming of cancer cells into regular-like cells for a complete cancer cure. The device includes an RF frequency generator for generating an RF signal, a low frequency generator for generating a modulating signal, a modulator for frequency modulating the RF signal with the modulating signal to produce a frequency-modulated output signal, and an antenna with a parabolic trough reflector (PTR) for directing the modulated signal to a patient's body.

According to an embodiment, an electromagnetic device for treating cancers and tumors can include a plurality of low frequency generators comprising a first low frequency generator configured for providing a first modulating signal having a modulation frequency of about 4 kHz, a second low frequency generator configured for providing a second modulating signal having a modulation frequency of about 7 kHz, a third low frequency generator configured for providing a third modulating signal having a modulation frequency of about 10 kHz, and a fourth low frequency generator configured for providing a fourth modulating signal having a modulation frequency of about 21 kHz; a radio frequency (RF) generator configured for providing an RF carrier signal having a frequency of 430 MHz; a modulator for frequency modulating the RF carrier signal with the modulating signals to produce a frequency-modulated output signal, an antenna; and a reflector connected to the antenna for directing the transmitted frequency-modulated output signal toward a patient's body.

A method for treating cancers and tumors, comprising providing a set of modulating signals using a plurality of low frequency generators, the set of modulating signals including a first modulating signal having a modulation frequency of about 4 kHz, a second modulating signal having a modulation frequency of about 7 kHz, a third modulating signal having a modulation frequency of about 10 kHz, and a fourth modulating signal having a modulation frequency of about 21 kHz; providing a radio frequency (RF) carrier signal having a frequency of 430 MHz using a radio frequency (RF) generator; frequency modulating the RF carrier signal with the set of modulating signals to produce a frequency-modulated output signal; and
directing the frequency-modulated output signal to a site of a cancer in a patient afflicted with the cancer.

In an embodiment, each modulation signal is applied in turn for a duration of about 30 minutes to provide a regular treatment regime and a total duration of the treatment session for the regular treatment regime is about 2 hours.

In an embodiment, each modulation signal is applied in turn for a duration of about 10 minutes and repeated two additional times to provide a tetragrammaton treatment regime, and a total duration of a tetragrammaton treatment regime is about 2 hours.

In an embodiment, a tetragramatton treatment regime is provided after the regular treatment regime. The tetragramatton treatment regime includes providing a second set of modulating signals including a first modulating signal having a modulation frequency of about 4 kHz, a second modulating signal having a modulation frequency of about 7 kHz, a third modulating signal having a modulation frequency of about 10 kHz, and a fourth modulating signal having a modulation frequency of about 21 kHz; providing a radio frequency (RF) carrier signal having a frequency of about 430 MHz using a radio frequency (RF) generator; frequency modulating the RF carrier signal with the second set of modulating signals to produce a frequency-modulated output signal; and directing the frequency-modulated output signal to a site of a cancer in a patient afflicted with the cancer, wherein each modulation signal is applied in turn for a duration of about 10 minutes to provide the second set of modulation signals, the second set of modulation signals is applied three times, and a total duration of a treatment session with the second set of modulation signals is about 2 hours.

These and other features of the present subject matter will become readily apparent upon further review of the following specification.

### BRIEF DESCRIPTION OF THE DRAWINGS

FIG. 1 is a perspective view of an electromagnetic (EM) device for treating cancers and tumors.

FIG. 2 is a schematic diagram of the of the EM device for treating cancers and tumors of FIG. 1.

FIG. 3 is a graph showing changes in the temperature of Sansevieria plant leaves as a function of time for exemplary modulation frequencies induced by the EM device for treating cancers and tumors of FIG. 1.

FIG. 4 is a plot showing changes in the temperature of squamous cell carcinoma (SCC) Cancer Cells as a function of time during irradiation by frequency-modulated RF electromagnetic waves generated by the EM device for treating cancers and tumors of FIG. 1.

FIG. 5A is a chart showing inhibition of neuroblastoma cancer cell (SHSY5Y) growth after irradiation by a frequency-modulated RF EM field generated by the EM device for treating cancers and tumors of FIG. 1.

FIG. 5B is a chart showing inhibition of SCC cancer cell (SCC47) growth after irradiation by a frequency-modulated RF EM field generated by the EM device for treating cancers and tumors of FIG. 1.

FIG. 6 is a plot of temperature changes of SCC cancer cells as a function of time, comparing temperature change during initial exposure to temperature change during a second exposure three hours later to a frequency-modulated RF EM field generated by the EM device for treating cancers and tumors of FIG. 1.

FIG. 7A is a chart showing inhibition of SCC cancer cell SCC47 growth after exposure to various frequency-modulated RF EM fields generated by the EM device for treating cancers and tumors of FIG. 1.

FIG. 7B is a chart showing inhibition of neuroblastoma cancer cell SHSY5Y growth after exposure to various frequency-modulated RF EM fields generated by the EM device for treating cancers and tumors of FIG. 1.

FIG. 8A is a chart showing inhibition of SCC cancer cell SCC47 cell growth after exposure to various frequency-modulated RF EM fields generated by the EM device for treating cancers and tumors of FIG. 1.

FIG. 8B is a chart showing inhibition of SCC cancer cell SCC47 and neuroblastoma cancer cell SHSY5Y growth after exposure to various frequency-modulated RF EM fields generated by the EM device for treating cancers and tumors of FIG. 1.

FIG. 9 is a graph of temperature changes of Sansevieria plant leaves as a function of power density of a frequency-modulated RF EM field generated by the EM device for treating cancers and tumors of FIG. 1.

FIG. 10 is a perspective view of an alternative embodiment of the electromagnetic (EM) device for treating cancers and tumors.

FIG. 11 is a schematic diagram of the EM device for treating cancers and tumors of FIG. 10.

FIG. 12 is a graph showing reprogramming efficiency versus RF power density for a set of frequencies.

FIGS. 13A-13C are Magnetic Resonance Imaging (MRI) brain scans of a patient suffering from glioma after (13A) one month; (13B) three months; and (13C) four and a half months of treatment using the EM device and method of the present teachings.

FIG. 14 is a graph showing efficiency of prostate adenoma treatment versus frequency of modulation.1

FIG. 15 is a graph showing efficiency of adenocarcinoma treatment versus frequency during treatment period.2

FIG. 16 is a graph showing efficiency of adenocarcinoma treatment versus frequency during treatment period.

FIG. 17 is a graph showing body temperature versus time at different modulation frequencies.4a

FIG. 18 is a graph showing body temperature versus time at different modulation frequencies.4b

FIG. 19 is a graph showing results of clinical trial for the prostate adenocarcinoma stage 1 treatment.5

FIG. 20 is a graph showing temperature measurements of the patient's body during treatment of IDC using the present method.

Similar reference characters denote corresponding features consistently throughout the attached drawings.

### DETAILED DESCRIPTION OF THE PREFERRED EMBODIMENTS

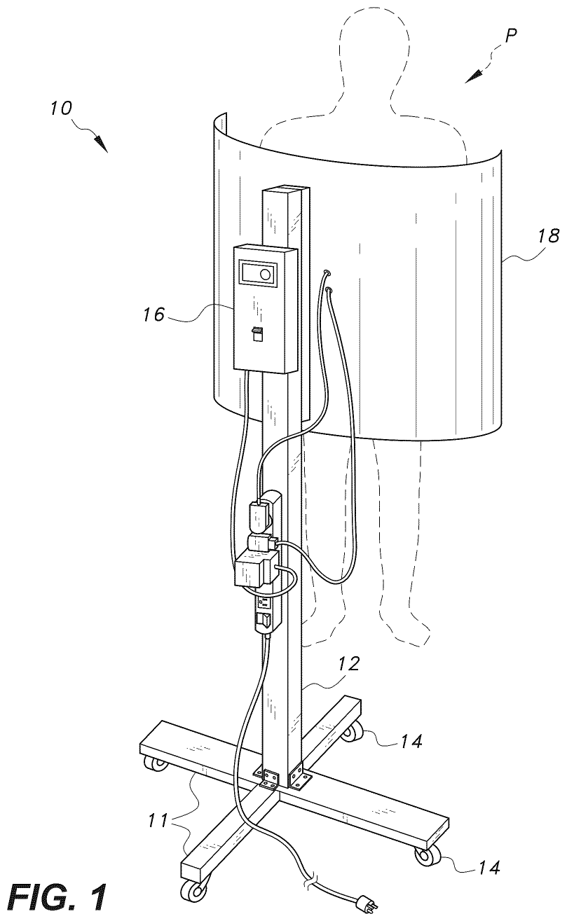

An embodiment of the electromagnetic (EM) device 10 for treating cancers and tumors is shown in FIG. 1. The device 10 includes a support holder or stand having a generally vertical support column 12 extending upward from the proximate end of four horizontal lower support members 11. The distal end of the horizontal lower support members 11 may include caster-type wheels 14 to allow the device 10 to be rolled into position for use or storage. The generally vertical support column 12 and the horizontal lower support members 11 may be made of aluminum. It should be noted that the holder for the device 10 may be of any suitable configuration, and the floor stand shown in FIG. 1 is for illustrative purposes only. The upper end of the vertical support column 12 has an electronic control box 16 and a parabolic trough reflector 18 mounted on opposite sides thereof. In use, a patient P is positioned at predetermined distance in front of the parabolic trough reflector 18 for receiving the RF energy from the device 10. For example, the predetermined distance may be between 0.5 m to 2 m.

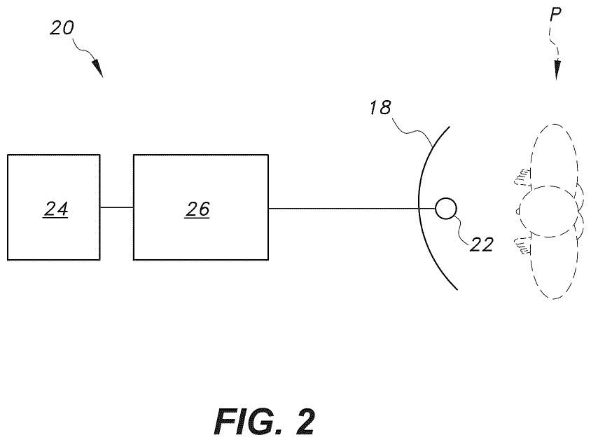

FIG. 2 shows a schematic diagram 20 of the EM device 10 of FIG. 1. The device 10 includes a low frequency generator 24 that provides a modulating signal of between 100 Hz to 10 kHz. The modulating signal is fed to block 26, which includes an FM modulator and a high frequency generator that provides an RF carrier signal in portions of the very high frequency (VHF) and ultra-high frequency (UHF) RF bands between 100 MHz and 900 MHz. The resulting FM RF signal is provided to an antenna element 22 at a power density of between 1 mW/cm² and 15 mW/cm² and is directed toward the patient P using the parabolic trough reflector 18. In addition to the above-described circuitry, the EM device 10 also includes one or more power supplies (not shown) for providing the required voltages for the various circuits, as is known in the art.

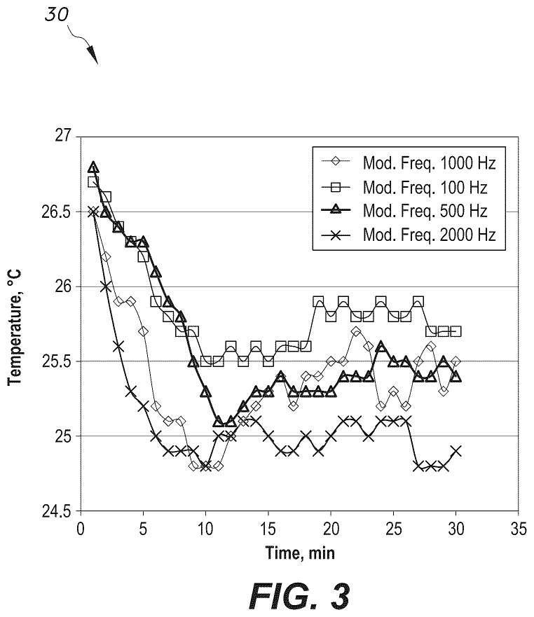

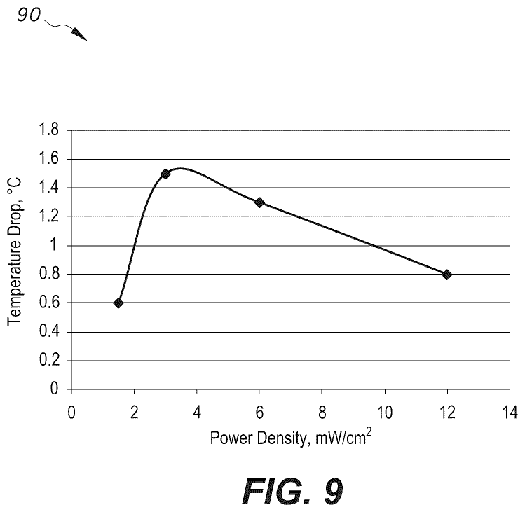

FIG. 3 is a graph 30 showing changes in the temperature of Sansevieria plant leaves for modulation frequencies of 100 Hz, 500 Hz, 1000 Hz and 2000 Hz of the FM RF EM waves from the EM device 10, the temperature being related to the metabolism of the Sansevieria plant leaves. It can be seen that for all of the modulation frequencies the plant temperature reduces over time due to exposure to the FM RF EM waves. FIG. 9 is a graph 90 of temperature change (and change in metabolism) of Sansevieria plant leaves as a function of power density of a frequency-modulated RF EM field generated by the EM device 10. The graph indicates that from a minimal power density of 1.5 mW/cm² to a power density of 3.5 mW/cm² there is an increasing amount of temperature drop of the Sansevieria plant leaves to a maximum temperature drop of 1.5° C. at a power density of 3.5 mW/cm². At power densities above 3.5 mW/cm² the temperature drop decreases in a linear manner. While not being bound by theory, this effect can be explained by considering two different physical processes. The first process works at low RF power density, when electrical forces induced by virtual photons interact with electrical fields of single nucleotides (where each single nucleotide is an electrical dipole) and this interaction decreases the Sansevieria plant cell temperature and metabolism. The second process starts to have a noticeable effect at higher power densities, when a large number of real photons results in partial absorption of these photons and their energy, and as result, above 3.5 mW/cm² the second process starts to offset the first process. With further increasing of the RF power density, the Sansevieria plant cell temperature will continue to increase until the temperature drop is a temperature rise.

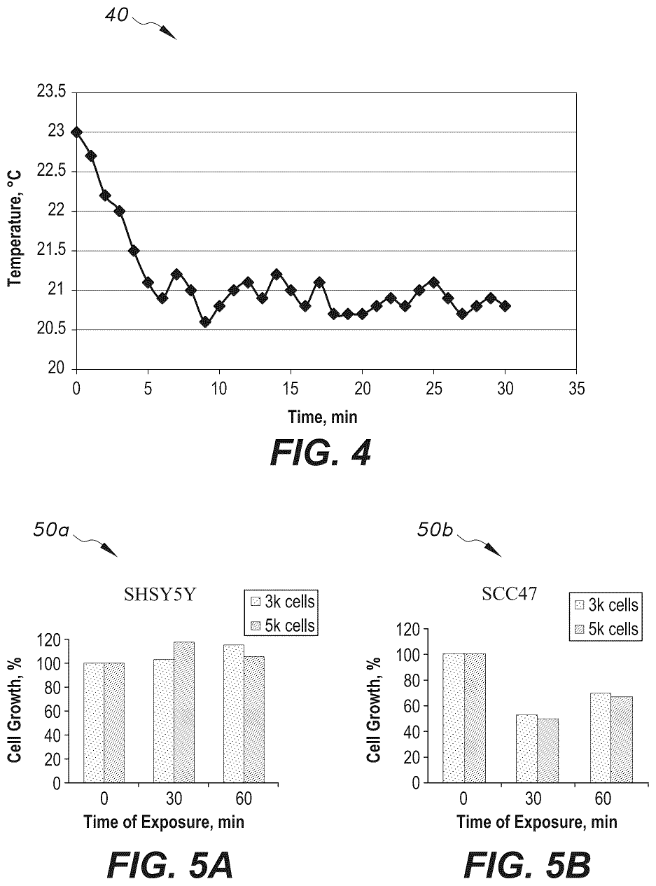

FIG. 4 is a plot 40 showing changes in the temperature of SCC cancer cells induced by FM RF EM waves from the EM device 10. The decreasing temperature of the SCC cancer cells over time indicates changes in the cells' metabolism during irradiation by the FM RF EM waves.

FIG. 5A is a chart 50a showing inhibition of neuroblastoma cancer cells (SHSY5Y) growth after exposure to an FM RF EM field generated by the EM device 10.

FIG. 5B is a chart 50b showing inhibition of SCC cancer cells (SCC47) growth after exposure to an FM RF EM field generated by the EM device 10. FIG. 5B shows clear inhibition of the SCC cancer cells growth after irradiation, which indicates the reprogramming of the SCC cancer cells.

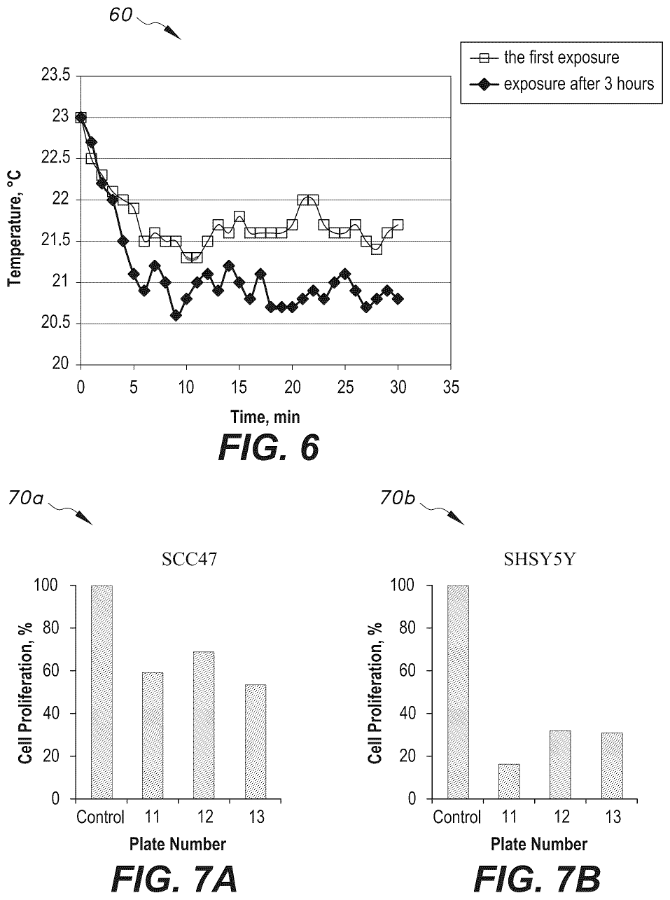

FIG. 6 is a plot 60 showing temperature changes of SCC cancer cells as a function of time during an initial 30-minute exposure to an FM RF EM field generated by the EM device 10 and during a second 30-minute exposure three hours later. The decreasing temperature of the SCC cancer cells over time indicates changes in the cells' metabolism during irradiation by the FM RF EM waves.

FIG. 7A is a chart 70a showing inhibition of SCC cancer cell (SCC47) growth after exposure to various frequency modulated RF EM fields from the EM device 10. On the x-axis, the control plate is for a sample that was not irradiated. Plate 11 indicates the reduced cell proliferation after a single 30-minute exposure to a 430 MHz RF carrier frequency modulated with a 1000 Hz modulating signal. Plate 12 indicates the reduced cell proliferation after a thirty-minute exposure including a first 10-minute exposure to a 430 MHz RF field frequency modulated with a 1000 Hz modulating signal, followed by a second 10-minute exposure to a 430 MHz RF field frequency modulated with a 500 Hz modulating signal, followed by a third 10-minute exposure to a 430 MHz RF field frequency modulated with a 200 Hz modulating signal. Plate 13 indicates the reduced cell proliferation after a first 30-minute exposure to a 430 MHz RF carrier frequency modulated with a 1000 Hz modulating signal, followed by a three-hour break between exposures, followed by a second 30-minute exposure to a 430 MHz RF carrier frequency modulated with a 500 Hz modulating signal.

FIG. 7B is a chart 70b showing inhibition of neuroblastoma cancer cell (SHSY5Y) growth after exposure to various frequency modulated RF EM fields generated by the EM device 10. On the x-axis, the plate designations are the same as in chart 70a of FIG. 7A.

FIGS. 7A and 7B show inhibition in the growth of cancer cells SCC47 (squamous cell carcinoma) and cancer cells SHSY5Y (neuroblastoma), which indicates a change in the cells' metabolism during irradiation using several different modulating frequencies and durations of FM RF EM fields generated by the EM device 10. From a comparison of charts 70a and 70b, it can be seen that there is a difference in reprogramming process efficiency for the two different kinds of the cancer cells (SCC47 and SHSY5Y) after the same irradiation time. This can be attributed to the proliferation speed of the SCC47 cells, which, in this case, was half the proliferation speed of the SHSY5Y cells.

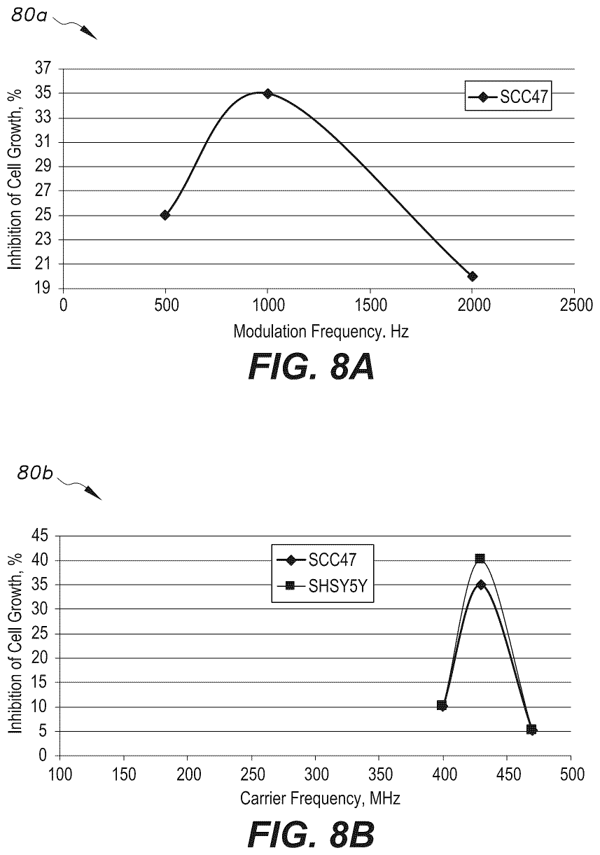

FIG. 8A is a chart 80a showing inhibition of SCC cancer cell SCC47 cell growth after exposure to various frequency-modulated RF EM fields generated by the EM device 10 with a modulation frequency from 500-2000 Hz. As can be seen from the chart 80a, the inhibition of the SCC cancer cells peaks at 35% with a 1000 Hz modulation frequency.

FIG. 8B is a chart 80b showing inhibition of SCC cancer cell SCC47 and neuroblastoma cancer cell SHSY5Y growth after exposure to various frequency-modulated RF EM fields generated by the EM device 10 with a carrier frequency from 400-470 Hz. As can be seen from the chart 80b, the inhibition of both types of cancer cells peaks at about a 430 Hz carrier frequency. As can also be seen from the chart 80b, the inhibition of the SCC cancer cells is 5% less than the inhibition of the neuroblastoma cancer cell at a 430 Hz carrier frequency. As previously noted, this can be attributed to the proliferation speed of the SCC47 cells, which in this case was half the proliferation speed of the SHSY5Y cells.

As previously noted, slow proliferation cancers require more irradiation procedures and more days of treatment in comparison with fast proliferation cancers. In order to increase the reprogramming efficiency to 100%, the irradiation process can be repeated several times in a 48-to-72-hour period. From testing, it has been found that the optimal number of 30-minute procedures for squamous cell carcinoma SCC47 cancer is twice a day with a 3-hour delay between procedures and requires repeating these procedures for four days. For neuroblastoma cancer cell SHSY5Y cancer, it was found that one 30-minute procedure per day for three days was adequate. The required number and schedule of irradiation procedures for maximum process efficacy can be calculated from the proliferation speed of the cancer cells and can be confirmed experimentally.

The present method can directly influence and reprogram the activity of single nucleotides inside cell genes at the time of cell proliferation. At the time of cell proliferation, live cells are very sensitive to any outside influence, even if this influence is very weak. The main difference between cancer cells and regular tissue cells is their frequency of proliferation. Cancer cells are immortal and proliferate without cease. Regular cells seldom proliferate and, even when they do, such proliferation is limited.

According to an embodiment, the present treatment method only impacts dividing cells at the time of cell proliferation. As such, administration of the present treatment avoids any damage to regular tissue cells during treatment. For this reason, the present method for treating cancers and tumors is safe for any patient except for pregnant women, as the cells of the fetus divide naturally and are very active.

The following parameters are very important for effectiveness of the present treatment: carrier frequency, frequency of modulation, depth of modulation, and RF power density.

High frequency RF generators create electromagnetic waves, which are directed by a metal reflector onto the patient's body. Low frequency generator use is for frequency modulation of the RF signal. Modulated RF signals induce fields of virtual photons in the patient's body. The field of each virtual photon has a size equal to that of a single nucleotide, which allows these fields to interact with each nucleotide with better efficiency.

Interaction between virtual photons and their own electrical field of nucleotides allows the possibility to directly influence the nucleotides' biological activity, as well as the possibility to cleave the connection between nucleotides at telomere ends at the time of cell proliferation and at a time when these connections are more sensitive to outside influence.

The distance between the antenna and the metal reflector can be equal to half of the wavelength. When a frequency of 430 MHz is used, corresponding to the wavelength 2=68 cm, the distance should be equal to 2/2=34 cm. In this case, photons reflected from the metal reflector have a phase shift of 180°, which makes it possible to create a field of virtual photons with a uniform distribution in space. This significantly increases the effectiveness and reliability of the cancer therapy.

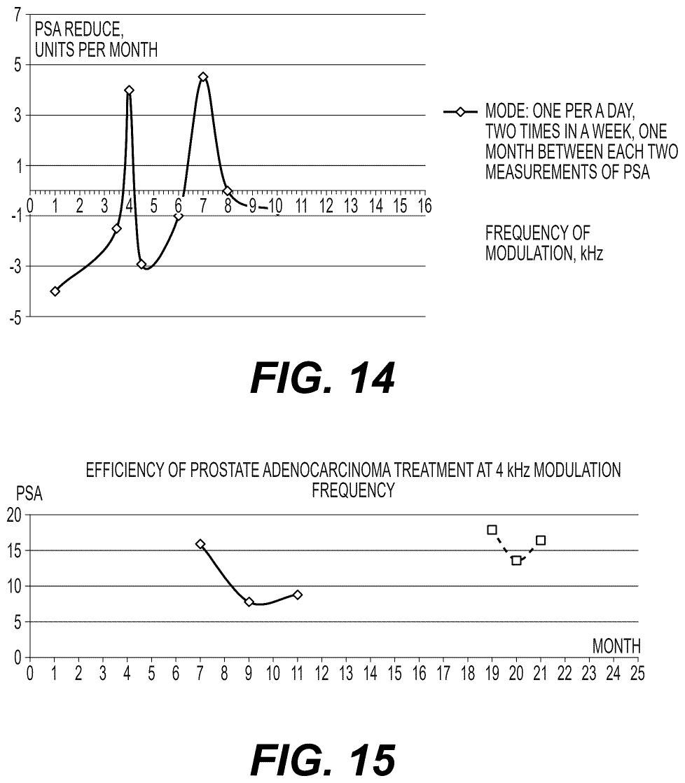

The present inventor has determined that an optimal frequency for cancer cell reprogramming is 430 MHz, as shown in FIG. 14, irrespective of the type of cancer that is being treated.

Frequency of modulation was tested in the treatment of prostate adenoma by measuring PSA once a month at different frequencies of the frequency modulation (FIG. 14). The possible optimal values for frequency modulation were 4 kHz and 7 kHz. It was discovered that the frequency of 4 kHz was ineffective for breast cancer Grade III treatment, but a 7 kHz frequency modulation showed a decrease in the size of the tumor from 5.5 cm to zero. This was within one month of beginning the treatment.

For testing depth of frequency modulation, all experiments were conducted at a depth of 80%.

For RF Power density, a metal reflector with an open aperture of 60 cm×60 cm with output RF power 10 W, gives a power density at a distance of 1 m equal to: 10/3600=3 mW/cm² or even less.

Telomeres are located at the ends of chromosomes and protect them from degradation. Suppressing the activity of telomerase, a telomere-synthesizing enzyme, and maintaining short telomeres is a protective mechanism against cancer in humans.

According to an embodiment, the present method includes delivering electrical fields of virtual photons to cancer cells during the proliferation of the cells, when connections between nucleotides inside genes are loose and may be easily severed.

According to an embodiment, the present method can inhibit the activity of telomerase and hTERT gene, which works as a catalyst for the telomere lengthening process. According to an embodiment, the present method can shorten telomere ends. In most human somatic cells, the expression of telomerase reverse transcriptase (hTERT) is repressed, and telomerase activity is inhibited. This leads to the progressive shortening of telomeres and inhibition of cell growth in a process called replicative senescence. Gene hTERT induction and telomerase activation not only create unlimited cancer cell proliferation potential by stabilizing telomere length (telomere lengthening-dependent) but also cause oncogenic effects independent of telomere lengthening function.

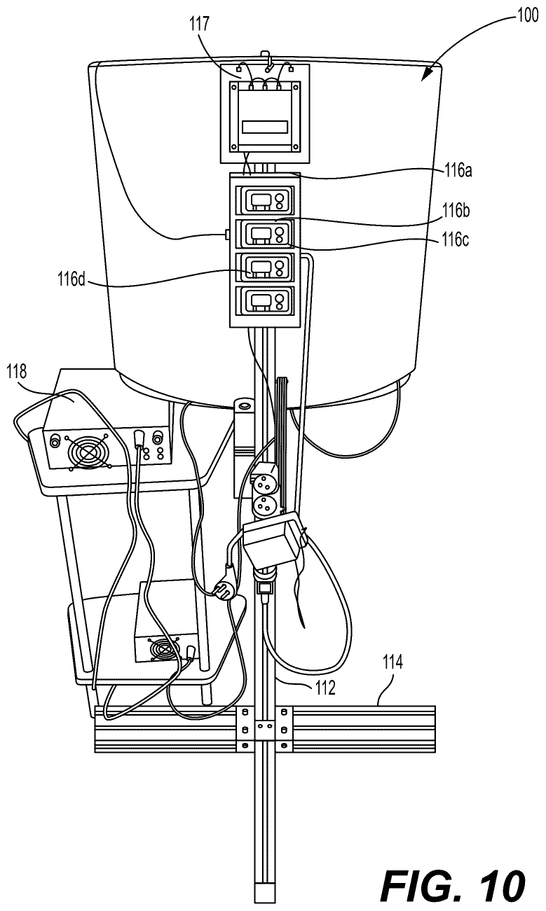

An alternative embodiment of the electromagnetic (EM) device is shown in FIG. 10, designated 100 in the drawings. Like the EM device 10, the EM device 100 includes a support holder or stand having a generally vertical support column 112 extending upward from the proximate end of four horizontal lower support members 114. The distal end of the horizontal lower support members 114 may include caster-type wheels (not shown) to allow the device 110 to be rolled into position for use or storage. The generally vertical support column 112 and the horizontal lower support members 114 may be made of aluminum. It should be noted that the holder for the device 110 may be of any suitable configuration, and the floor stand shown in FIG. 10 is for illustrative purposes only. The device 100 includes a plurality of low frequency generators 116a-116d, a radio frequency (RF) generator 118, and a programmable timer 117. A parabolic trough reflector is mounted on the vertical support column 112. In use, a patient P is positioned at a predetermined distance in front of the parabolic trough reflector for receiving the RF energy from the device 100. For example, the predetermined distance may be between 0.5 m to 2 m, such as 1 m.

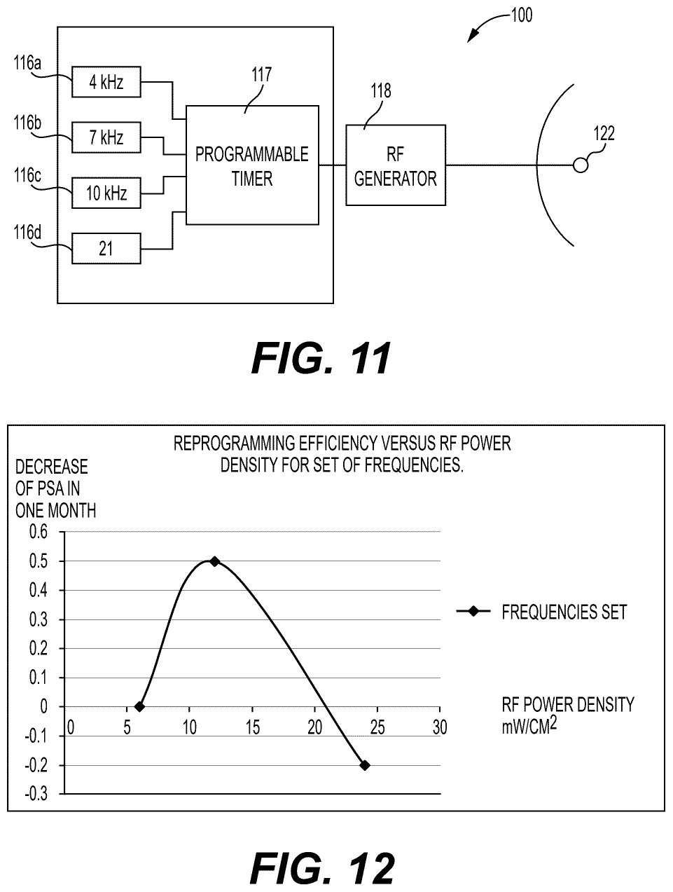

FIG. 11 shows a schematic diagram 100 of the EM device 100 of FIG. 10. Each of a plurality of low frequency generators 116a-116d provide, in turn, a modulation signal to a radio frequency (RF) generator 118 for providing an RF carrier signal and frequency modulating the RF carrier signal with the modulating signals to produce a frequency-modulated output signal. The low frequency generators 116a-116d include a first low frequency generator 116a which provides a first signal having a modulation frequency of about 4 kHz, a second low frequency generator 116b which provides a second signal having a modulation frequency of about 7 kHz, a third low frequency generator 116c which provides a third signal having a modulation frequency of about 10 kHz, and a fourth low frequency generator 116d which provides a fourth signal having a modulation frequency of about 21 kHz. A programmable timer 117 can be programmed to ensure that each of the modulation signals are applied in turn and for a desired duration.

In one embodiment, a method for treating cancers and tumors can include administering a regular treatment regime to the patient using the EM device 100. The regular treatment regime can include generating a first set of modulation signals using the plurality of low frequency generators. In an embodiment, a first set of modulation signals can include a first modulation signal having a modulation frequency of about 4 kHz, a second modulation signal having a modulation frequency of about 7 kHz, a third modulation signal having a modulation frequency about 10 kHz, and a fourth modulation signal having a modulation frequency about 21 kHz. The method can further include providing a radio frequency (RF) carrier signal having a frequency of 430 MHz using a radio frequency (RF) generator; frequency modulating the RF carrier signal with the set of modulating signals to produce a frequency-modulated output signal; and directing the frequency-modulated output signal to a site of a cancer in a patient afflicted with the cancer. In an embodiment, each modulation signal can be applied in turn for a duration of about 30 minutes. Thus, a total duration of the full treatment for the regular regime can be about 2 hours.

In one embodiment, a method for treating cancers and tumors can include administering a tetragrammaton treatment regime to the patient. The tetragrammaton treatment regime can include generating a first set of modulation signals using a plurality of low frequency generators. In an embodiment, a first set of modulation signals can include a first modulation signal having a modulation frequency of about 4 kHz, a second modulation signal having a modulation frequency of about 7 kHz, a third modulation signal having a modulation frequency of about 10 kHz, and a fourth modulation signal having a modulation frequency of about 21 kHz; providing a radio frequency (RF) carrier signal having a frequency of 430 MHz using a radio frequency (RF) generator; frequency modulating the RF carrier signal with the set of modulating signals to produce a frequency-modulated output signal; and directing the frequency-modulated output signal to a site of a cancer in a patient afflicted with the cancer. In an embodiment, each modulation signal can be applied in turn for a duration of about 10 minutes and repeated twice to complete the full treatment. Thus, a total duration of the full treatment for the tetragrammaton regime can be about 2 hours.

In another embodiment, a regular treatment regime can be followed by a tetragrammaton treatment regime to provide a combination treatment. Thus, after administering the regular treatment regime, a second set of modulation signals can be applied including a first modulation signal having a modulation frequency of about 4 kHz, a second modulation signal having a modulation frequency of about 7 kHz, a third modulation signal having a modulation frequency of about 10 kHz, and a fourth modulation signal having a modulation frequency of about 21 kHz. A radio frequency (RF) carrier signal can be provided having a frequency of about 430 MHz using a radio frequency (RF) generator. The RF carrier signal can be frequency modulated with the second set of modulating signals to produce a frequency-modulated output signal; and the frequency-modulated output signal can be directed to a site of a cancer in a patient afflicted with the cancer, wherein each modulation signal is applied in turn for a duration of about 10 minutes to provide the second set of modulation signals. The second set of modulation signals under the tetragrammaton regime can be applied three times during a treatment session.

In an embodiment, the combined regime can include one month of cancer treatment in accordance with the regular regime, then two weeks of cancer treatment in accordance with tetragrammaton regime, followed by providing the regular regime for at least another month. Using such a combined regime allows a significant decrease of the initial body temperature and can serve to cure the cancer faster.

In some embodiments, it is preferable not to use the tetragrammaton regime independently as this regime can increase the prostate specific antigen (PSA) levels by 5 units after only two weeks of the treatment. Thus, it can be preferable to use the tetragrammaton treatment regime as part of a combined treatment regime. For example, the tetragrammaton regime can be followed with the regular regime for one month or longer in order to decrease the PSA levels back to the original levels.

In an embodiment, the frequency-modulated output signal can be directed to a site of a cancer in a patient from a distance of about 1 meter from the patient. For example, a distance from the antenna element to the patient (P) can be about 1 meter, the frequency-modulated output signal is applied at an RF power density less than about 12 mW/cm². RF power density is an important factor because at a power density less than 12 mW/cm², the present method can be more efficient than powers between 12 mW/cm² and 24 mW/cm² or a power of 6 mW/cm² or below level (FIG. 12).

In addition to the above-described circuitry, the EM device 100 also includes one or more power supplies (not shown) for providing the required voltages for the various circuits, as is known in the art.

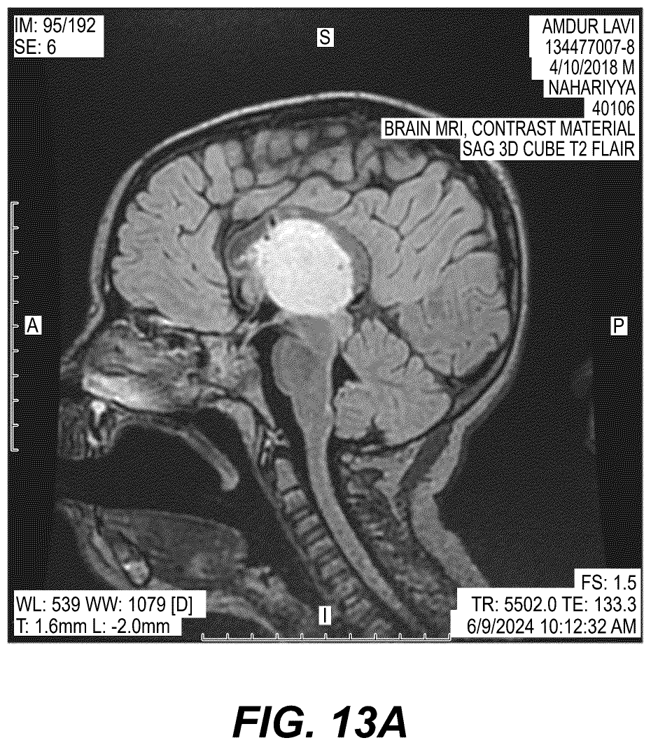

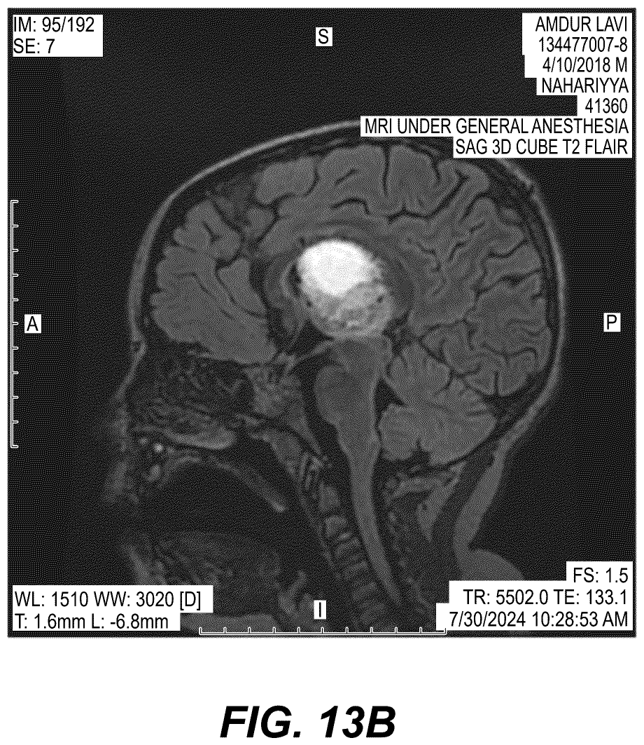

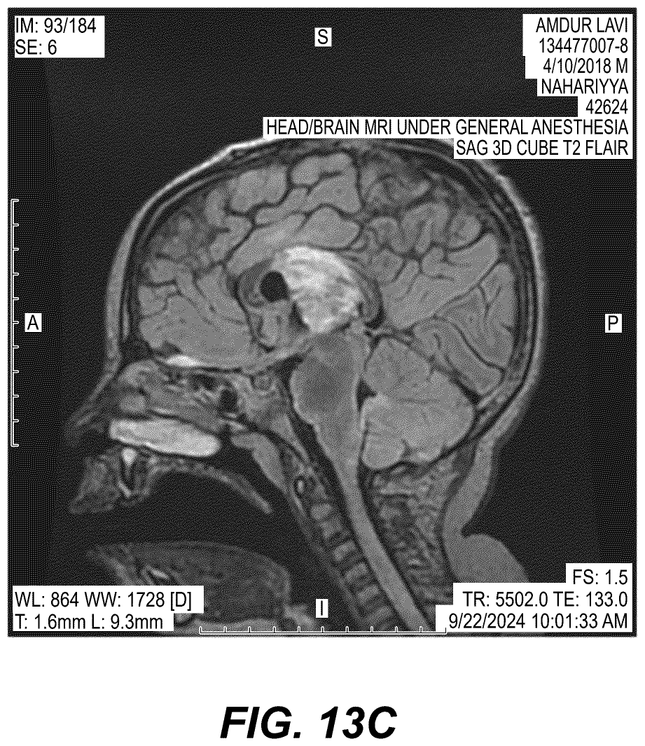

In an embodiment, an appropriate time delay between each of two treatment sessions should be maintained and can be different for different cancer types. The time delay can depend upon the proliferation speed of the cancer cells. For example, for treatment of prostate adenocarcinoma, the optimal time delay can be about 72 hours (2 times in a week). For treatment of Triple Negative Invasive Ductal Carcinoma, an optimal Time Delay can be 48 hours (3 times in a week). For treatment of Glioma and Glioblastoma, an optimal Time Delay be 24 hours (once a day). FIGS. 13A-13C show Magnetic Resonance Imaging (MRI) brain scans of a patient suffering from glioma after (13A) one month; (13B) three months; and (13C) four and a half months of treatment using the EM device 100.

In an embodiment, an optimal time delay for a cancer treatment can be determined by measuring the initial body temperature of a patient before each session. If the initial temperature is higher than 36.6° C. and decreases after the next several sessions, e.g., temperature measurements approach 36.6° C. or less, this indicates an optimal time delay. However, if the initial temperature stabilizes at a higher level, for example 36.9° C., it suggests that the time interval between sessions should be reduced in order to enhance the likelihood of a complete cure.

As a result of the death of cancer cells inside the tumor, bleeding may occur. In order to speed up the treatment process and to avoid any negative side effects, it is preferable to take a break after each month of the treatment. For example, in the case of a short interval between treatment sessions (less than 48 hours) it is recommended to take a break of about 4-7 days after each month of the treatment.

In an embodiment, the method includes taking body temperature measurements of the patient before and after each treatment session. Regular body temperature measurements can facilitate monitoring cancer treatment progress and cancer treatment efficiency. In an embodiment, body temperature measurements before and after each treatment session can be used for live cancer cell detection. This method of cancer cell detection is comparable with the PET-CT test.

According to an embodiment, the present method can be used to treat a cancer selected from the group consisting of glioma, lung adenocarcinoma, endometrioid adenocarcinoma, prostate adenocarcinoma and invasive ductal carcinoma. In an embodiment, the cancer is selected from the group consisting of mucinous lung adenocarcinoma (Grade IV), endomitroidal adenocarcinoma (Grade IV).

According to an embodiment, a full cancer treatment process is complete if the body temperature after a treatment session is 0.5° C. lower than the initial temperature during about five to six treatment sessions.

According to an embodiment, the present method can be used for cancer treatment by irradiation of RF waves at frequency (f=430 MHz), corresponding to the wavelength 2=0.68 m, to generate virtual photons according to the equation:

λ=k·λv, (1),

where k=2·109 is the dimensionless coefficient, and λv is wavelength of virtual photons. In this case λv=0.34 nm.

If the bandwidths of generated RF signal fbw is equal to: fbw=25 kHz, then virtual photons will be generated in the wavelength range: from f−fbw/2 to f+fbw/2. When the frequency modulation (fm) is used, the wavelength range will be equal to:

From (f−fbw/2)−fm to (f+fbw/2)+fm (2)

If the carrier frequency f=430 MHz, bandwidth fbw=25 kHz and frequency of modulation fm=5 kHz, then modulated signal will be in the range: 429.983 MHz-430.175 MHz, or 0.6974 m−0.6977 m=0.0003 m=3.10-4 m, what corresponds to pulsation of static electric field of virtual photons in the volume in the range 6·10-13 m. If frequency of modulation is equal to 20 kHz, then pulsation of static electric field of virtual photons in the volume are in the range 24·10-13 m.

According to an embodiment, FM modulation can be used in the present method for treating cancers and tumors. Frequency modulated RF waves generate static electric fields with amplitude of pulsations from 6·10-13 m to 24·10-13 m in the volume. These amplitudes are very small, but they still can have an influence on connections between nucleotides at the time of their proliferation and even enough to disconnect (to cleave) them.

When using FM modulation, the electric field of virtual photons can be changed not only in space but also in time. The period of these pulsations depends on the frequency of modulation. For example, when fm=4 kHz, the duration of one pulsation is equal to: T1=0.25 ms, and if fm=20 kHz, then T2=0.05 ms. The process of cancer cell division can occur over some time, when connections at the ends of telomeres of two diverging new cells are subject to increasing tension. These connections can be cleaved under external influences by a weak static electric field. In such a case, periods T1 and T2 can be the interaction periods to cleave nucleotide connections. They will depend on the speed of cancer cell proliferation. Experimental tests confirmed these predictions.

It is believed that the present method inhibits the activity of telomerase and the telomerase reverse transcriptase (hTERT) gene which works as a catalyst for the telomere lengthening process. According to an embodiment, the present method can kill cancer cells quickly by shortening telomere ends. In an embodiment, the method can include applying electrical fields of the virtual photons to the cells during cell proliferation when connections between nucleotides inside genes are loose and may be easily severed.

According to an embodiment, the present method can cure metastatic cancer, or cancer Stage IV completely. As described herein, the present method applies frequency-modulated RF irradiation to the patient's body. In this process, any cancer cell in the body can be reprogrammed at the time of cell proliferation into normal cells and then proceed to apoptosis. Because all cancer cells cannot proliferate at the same time, treatment sessions can be repeated until the cancer is cured completely.

According to an embodiment, a full cancer treatment process is complete if the body temperature after a treatment session is 0.5° C. lower than the initial temperature during about five to six treatment sessions.

According to an embodiment, a lowered body temperature of a patient after a treatment session according to the present teachings can indicate successful treatment of the cancer. According to an embodiment, when the body temperature after each treatment session is lower than an initial body temperature or temperature prior to treatment and continues to be lower after several subsequent

According to an embodiment, adenocarcinoma and invasive ductal carcinoma can be treated using the following set of optimal modulation frequencies during one treatment session: 4 kHz, 7 kHz, 10 kHz, and 21 kHz.

The present teachings are illustrated by the following examples.

### Example 1

### Breast Cancer

A breast tumor with an initial size of 5.5 cm was decreased, cured, and finally disappeared completely during the first month of treatment using the EM device 100 at a frequency modulation of 7 kHz. A full and complete cancer treatment was conducted using a full set of optimal modulation frequencies.

The protocol of the full cancer treatment was as follows. Carrier frequency was 430 MHz. Frequencies of modulation were 4 KHz, 7 kHz, 10 kHz and 21 kHz. Each frequency session was 30 min. One full treatment session was 2 hours. RF power density was less than 12 mW/cm² at a distance from the antenna of approximately 1 m. The time delay between treatment sessions was 48 hours (three times in a week).

After the tumor disappeared, the experiment was continued with body temperature measurements taken both before and after each treatment session. At the conclusion of the experiment, during the final seven sessions, the body temperature was lower than the initial body temperature before the session commenced.

Each modulation frequency was applied for 30 minutes and the time period between each two treatment sessions was 48 hours.

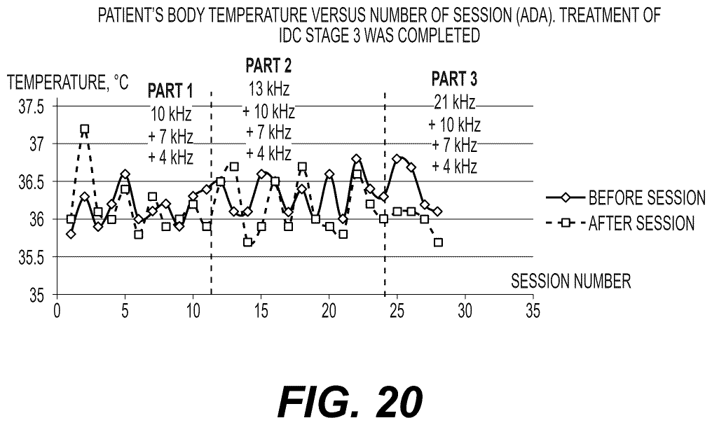

As one can see from temperature measurements in Part 3 of FIG. 20, initial body temperatures were higher than temperatures after the treatment session during all of the seven final measurements. This means that the cancer was cured completely. As can be seen from FIG. 20, the optimal frequencies of modulation for effective treatment of Triple-Negative IDC cancer were as follows: 4 kHz, 7 kHz, 10 kHz and 21 kHz, applied in turn, for the best and fastest anti-cancer treatment effect. Because this kind of cancer is very aggressive, effective treatment requires using three treatment sessions per week or every 48 hours. The entire process to cure cancer completely took three months.

For all patients with cancer, which were cured by the present method, a temperature test was taken every six months, as described above. If the initial body temperature was lower than after the treatment session (even once), the treatment was continued. The results of a PET-CT scan, performed one year after the start of this cancer treatment with the present method, showed a complete absence of any sign of cancer. Further, no negative side effects were observed either during or after the cancer treatment period.

### Example 2

### Prostate Adenocarcinoma

Effectiveness of treatment of prostate adenocarcinoma using the present method was demonstrated, as shown in FIG. 14. A 65-year old male patient suffering from prostate adenocarcinoma was treated according to the following clinical protocol: frequency 430 MHz, RF power density 12 mW/cm², distance equal to 1 m, each session being 30 min long, once a day, twice a week. Levels of PSA were measured each month, once a month.

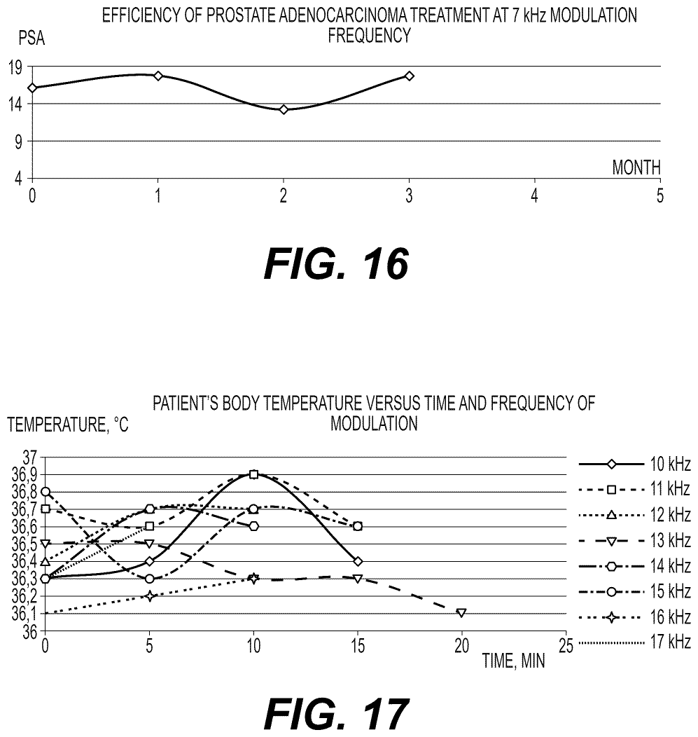

Because of the existence of at least two different optimal modulation frequencies for treatment of prostate adenocarcinoma, it follows that in one prostate adenocarcinoma tumor, there are at least two different types of cancer cells that differ in the rate of their division. This conclusion was confirmed by the experimental results shown in FIG. 15 and FIG. 16. In both described cases, the PSA value decreased after the start of the therapy and then returned to its previous value. Accordingly, initially, at the start of therapy at 4 kHz, the MRI test showed PIRADS 3 and signs of cancer. A biopsy test indicated that in one probe out of a total of 6 probes, there were 25% prostatic adenocarcinoma cells in the volume of this probe. After finishing the treatment, MRI test results showed PIRADS 1 and no signs of cancer. An MRI test carried out one year later, showed PIRADS 4 again and the presence of cancer in the area with size 0.7 cm. The next biopsy test from this area indicated that in one probe out of a total of 7 probes, there was less than 5% prostatic adenocarcinoma cells by volume within this probe. As can be seen from FIG. 16, using frequency of modulation 7 kHz leads to the same therapy result. From all these results, it follows that the use of only one frequency of modulation in the prostate adenocarcinoma treatment can lead to apoptosis of only one part of the cancer cells, while all other parts of the cancer cells continue proliferation without dying.

All of these results were confirmed by the next clinical trial for treatment of Invasive Ductal Carcinoma (IDC), which was cured completely. In this case, the first step of the therapy was treatment at a modulation frequency of 4 kHz during an initial three-month period. After this, the second step was treatment at a modulation frequency of 7 kHz during another two-month period and the third step was treatment at a modulation frequency of 10 kHz during a final two-month period. An experiment was conducted to find additional optimal modulation frequencies at frequencies 4 kHz and 7 kHz (results shown in FIG. 14). A reduction of cell metabolism is part of the reprogramming process. This will provide maximum reduction in body temperature (and the corresponding intensity of the cell metabolism) and should be tested for cancer cells during the reprogramming process. Results of this experiment are shown in FIG. 17.

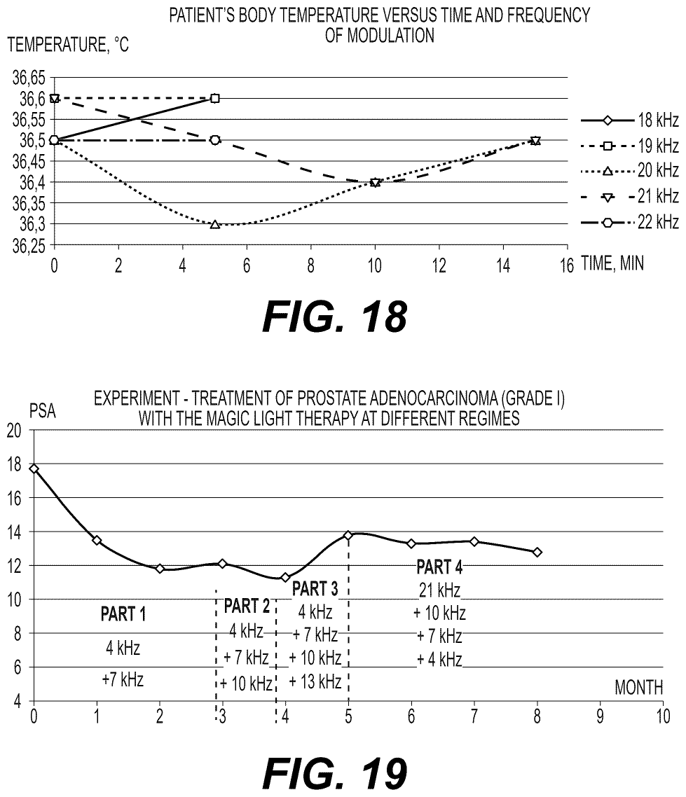

As one can see in FIG. 17, the modulation frequency of 13 kHz is an optimal choice for testing the cell reprogramming process because at this frequency, reduction in cell metabolism was maximal. As one can see in FIG. 18, the modulation frequency of 21 kHz is also an optimal choice to test the cell reprogramming process because at this frequency, the reduction in cell metabolism was maximal and the process of reducing the metabolism was longer.

In the clinical trial for the treatment of prostate adenocarcinoma, the present method was tested on prostate adenocarcinoma in the following manner. Part 1 included two different frequencies of modulation in turn, in one session. Part 2 included three different modulation frequencies, in turn, during each session. Part 3 and Part 4 included four different modulation frequencies, in turn, during each session. Part 1 was at frequencies 4 kHz and 7 kHz. Part 2 was at frequencies 4 kHz, 7 kHz and 10 kHz. Part 3 was at frequencies 4 kHz, 7 kHz, 10 kHz and 13 kHz. Part 4 was at frequencies 4 kHz, 7 kHz, 10 kHz and 21 kHz. The result of this clinical trial is presented in FIG. 19.

Part 1 clinical protocol included a carrier frequency of 430 MHz, frequency of modulation was 4 kHz for 30 minutes, 7 kHz for 30 minutes, once a day, three times a week, 15 sessions in a month. The RF Power Density was 12 mW/cm² and a distance to the patient's body was equal to 1 m. PSA was measured once a month.

Part 2 clinical protocol included a carrier frequency of 430 MHz, frequency of modulation was 4 kHz for 25 minutes, then 7 kHz for 25 minutes, then 10 kHz for 25 min, once a day, three times a week, with 15 such sessions in a month. The RF Power Density was 12 mW/cm², and a distance from the antenna to the patient's body was equal to 1 m. PSA was measured once a month.

Part 3 clinical protocol included a carrier frequency of 430 MHz, frequency of modulation was 4 kHz for 25 minutes, then 7 kHz also for 25 minutes, then 10 kHz for 25 minutes, then 13 kHz for 25 min, once a day, three times in a week. Fifteen sessions were administered in this manner for one month. The RF Power Density was 12 mW/cm², and a distance from the antenna to the patient body was equal to 1 m. PSA was measured once a month.

Part 4 clinical protocol included a carrier frequency of 430 MHz, frequency of modulation was 4 kHz for 30 minutes, then 7 kHz for 30 minutes, then 10 kHz for 30 minutes, then 21 kHz for 30 min, once a day, twice a week. Ten treatment sessions were administered in this manner for one month. The RF Power Density was 12 mW/cm² and the distance from the antenna to the patient body was 1 m. PSA was measured once a month.

It is to be understood that the electromagnetic device and method for treating cancers and tumors is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. An electromagnetic device for treating cancers and tumors, comprising:
a plurality of low frequency generators comprising a first low frequency generator configured for providing a first modulating signal having a modulation frequency of about 4 kHz, a second low frequency generator configured for providing a second modulating signal having a modulation frequency of about 7 kHz, a third low frequency generator configured for providing a third modulating signal having a modulation frequency of about 10 kHz, and a fourth low frequency generator configured for providing a fourth modulating signal having a modulation frequency of about 21 kHz;
a radio frequency (RF) generator configured for providing an RF carrier signal having a frequency of 430 MHz;
a modulator for frequency modulating the RF carrier signal with the modulating signals to produce a frequency-modulated output signal,
an antenna; and
a reflector connected to the antenna for directing the transmitted frequency-modulated output signal toward a patient's body.

2. The electromagnetic device of claim 1, wherein the reflector is a parabolic trough reflector.

3. A method for treating cancers, comprising using the electromagnetic device of claim 1 for directing a frequency-modulated output signal produced by the modulator to a site of a cancer in a patient afflicted with the cancer.

4. The method for treating cancers according to claim 3, wherein the step of directing the frequency-modulated output signal comprises directing the frequency-modulated output signal to a site of a cancer in a patient from a distance of about 1 meter from the patient.

5. The method for treating cancers according to claim 3, wherein the frequency-modulated output signal is applied at an RF power density less than about 12 mW/cm².

6. The method for treating cancers according to claim 3, wherein the frequency-modulated output signal is applied at an RF power density ranging from about 6 mW/cm² to about 12 mW/cm².

7. The method for treating cancers according to claim 6, wherein the modulator provides the frequency-modulated output signal by frequency modulating an RF carrier signal with a set of modulating signals, the set of modulating signals including a first modulation signal having a modulation frequency of about 4 kHz, a second modulation signal having a modulation frequency of about 7 kHz, a third modulation signal having a modulation frequency of about 10 kHz, and a fourth modulation signal having a modulation frequency of about 21 kHz, wherein each modulation signal is applied in turn for a duration of about 30 minutes and a total duration of a treatment session is about 2 hours.

8. The method for treating cancers according to claim 6, wherein the modulator provides the frequency-modulated output signal by frequency modulating an RF carrier signal with a set of modulating signals, the set of modulating signals including a first modulation signal having a modulation frequency of about 4 kHz, a second modulation signal having a modulation frequency of about 7 kHz, a third modulation signal having a modulation frequency of about 10 kHz, and a fourth modulation signal having a modulation frequency of about 21 kHz, wherein each modulation signal is applied in turn for a duration of about 10 minutes, the set of modulation signals is applied three times during a treatment session, and a total duration of the treatment session is about 2 hours.

9. The method for treating cancers according to claim 6, further comprising measuring a body temperature of the patient before directing a frequency-modulated output signal produced by the modulator to a site of a cancer in a patient afflicted with the cancer.

10. The method for treating cancers according to claim 6, further comprising measuring a body temperature of the patient before and after directing a frequency-modulated output signal produced by the modulator to a site of a cancer in a patient afflicted with the cancer.

11. A method for treating cancers and tumors, comprising:
providing a set of modulating signals using a plurality of low frequency generators, the set of modulating signals including a first modulating signal having a modulation frequency of about 4 kHz, a second modulating signal having a modulation frequency of about 7 kHz, a third modulating signal having a modulation frequency of about 10 kHz, and a fourth modulating signal having a modulation frequency of about 21 kHz;
providing a radio frequency (RF) carrier signal having a frequency of 430 MHz using a radio frequency (RF) generator;
frequency modulating the RF carrier signal with the set of modulating signals to produce a frequency-modulated output signal; and
directing the frequency-modulated output signal to a site of a cancer in a patient afflicted with the cancer.

12. The method of claim 11, wherein each modulating signal is applied in turn for a duration of about 30 minutes to provide a regular treatment regime and a total duration of a treatment session is about 2 hours.

13. The method of claim 12, further comprising:
providing a second set of modulating signals using a plurality of low frequency generators, the second set of modulating signals including a first modulating signal having a modulation frequency of about 4 kHz, a second modulating signal having a modulation frequency of about 7 kHz, a third modulating signal having a modulation frequency of about 10 kHz, and a fourth modulating signal having a modulation frequency of about 21 kHz;
providing a radio frequency (RF) carrier signal having a frequency of about 430 MHz using a radio frequency (RF) generator;
frequency modulating the RF carrier signal with the second set of modulating signals to produce a frequency-modulated output signal; and
directing the frequency-modulated output signal to a site of a cancer in a patient afflicted with the cancer, wherein
each of the second set of modulating signals is applied in turn for a duration of about 10 minutes to provide the second set of modulating signals,
the second set of modulating signals is applied three times, and
a total duration of a treatment session with the second set of modulating signals is about 2 hours.

14. The method of claim 11, wherein each of the second set of modulating signals is applied in turn for a duration of about 10 minutes and repeated two additional times to provide a tetragrammaton treatment session, a total duration of a tetragrammaton treatment session being about 2 hours.

15. The method for treating cancers according to claim 11, wherein the step of directing the frequency-modulated output signal comprises directing the frequency-modulated output signal to a site of a cancer in a patient from a distance of about 1 meter from the patient.

16. The method for treating cancers according to claim 11, wherein the frequency-modulated output signal is applied at an RF power density less than about 12 mW/cm².

17. The method for treating cancers according to claim 11, wherein the frequency-modulated output signal is applied at an RF power density ranging from about 6 mW/cm² to about 12 mW/cm².

18. A method of treating cancers in a patient, comprising applying frequency-modulated RF irradiation to the body of the patient, the frequency-modulated RF irradiation comprising a modulation signal and a radio frequency (RF) carrier signal having a frequency of 430 MHz, wherein the cancer is selected from the group consisting of glioma, lung adenocarcinoma, endometrioid adenocarcinoma, prostate adenocarcinoma and invasive ductal carcinoma.

19. The method of claim 18, wherein the frequency-modulated RF irradiation shortens telomere ends.

20. The method of claim 18, wherein the modulation signal comprises a plurality of modulation signals including a first modulation signal having a modulation frequency of about 4 kHz, a second modulation signal having a modulation frequency of about 7 kHz, a third modulation signal having a modulation frequency about 10 kHz, and a fourth modulation signal having a modulation frequency about 21 kHz.
